(19) United States
(12) Patent Application Publication
(10) Pub. No.: US 2014/0268038 A1
(43) Pub. Date: Sep. 18, 2014

(54) SYSTEMS AND METHODS FOR VARIABLE DEPTH OPTICAL COHERENCE TOMOGRAPHY IMAGING

(71) Applicant: CARL ZEISS MEDITEC, INC., Dublin, CA (US)

(72) Inventor: Tilman SCHMOLL, Dublin, CA (US)

(73) Assignee: CARL ZEISS MEDITEC, INC., Dublin, CA (US)

(21) Appl. No.: 13/795,953

(22) Filed: Mar. 12, 2013

(51) Int. Cl. A61B 3/10

(52) U.S. Cl. CPC A61B 3/102
USPC 351/206; 351/246

(57) ABSTRACT

Systems and methods for variable depth optical coherence tomography (OCT) imaging with a swept laser source are presented. Swept-source OCT data acquired at two different spectral resolutions is resampled using information on the wavenumber nonlinearity of the source to generate OCT images at two different depths.

### TECHNICAL FIELD

One or more embodiments of the present invention relate generally to improvements in Optical Coherence Tomography (OCT) systems, methods, and applications. In particular it is an object of the present invention to enable swept-source OCT imaging with adjustable imaging depth.

### BACKGROUND

Optical coherence tomography (OCT) is a noninvasive, noncontact imaging modality that uses coherence gating to obtain high-resolution cross-sectional images of tissue microstructure. In Frequency domain OCT (FD-OCT), the interferometric signal between light from a reference and the back-scattered light from a sample point is recorded in the frequency domain rather than the time domain. After a wavelength calibration, a one-dimensional Fourier transform is taken to obtain an A-line spatial distribution of the object scattering potential. The spectral information discrimination in FD-OCT can be accomplished by using a dispersive spectrometer in the detection arm in the case of spectral-domain OCT (SD-OCT) or rapidly tuning a swept laser source in the case of swept-source OCT (SS-OCT).

The axial or depth resolution of the FD-OCT system is determined by the actual spectral width recorded and used for reconstruction. The axial range over which an OCT image is taken (imaging depth, scan depth or imaging range) is determined by the sampling interval or resolution of the optical frequencies recorded by the OCT system. Different applications of OCT require different imaging depths. For instance while for imaging of the posterior eye a depth of 2 to 4 mm is sufficient, imaging the anterior eye from cornea to lens requires at least 10 mm imaging depth. Variable depth imaging can be realized by varying the spectral resolution. This variability is difficult to introduce with SD-OCT as the spectrometer resolution determines the spectral resolution. Specifically, in SD-OCT, the spectrometer disperses different wavelengths to the detector elements. The resolution of the optical frequencies and therefore the imaging depth depends on the width of the portion of the spectrum that is measured by a single detector element or pixel.

In SS-OCT the spectral resolution is controlled by the instantaneous line width of the swept source as well as the sampling rate in conjunction with the sweep rate (see for example Yun et al. “High-speed optical frequency-domain imaging” Optics Express 11(22), 2953-2963 (2003)). The spectral resolution of the SS-OCT system can therefore be changed to acquire data of two different depth ranges by modifying either one of these parameters. In SS-OCT, the sweep is often not linear in wavenumber, k, so in many cases a reference interferometer is used to establish a reference signal, which yields information of the swept source's wavenumber non-linearity. When this information is used to trigger the sampling it is often referred to as a “k-clock”. The reference interferometer then defines the sampling rate and different k-clocks could be used for different imaging depths as described in U.S. patent application Ser. No. 13/354,066 filed on Jan. 19, 2012. Alternatively the information of the swept source's wavenumber non-linearity may be used to resample data acquired linearly in time to be sampled linearly in wavenumber.

### SUMMARY

Here we propose using linear temporal acquisition in combination with numerical resampling of the data for a variable depth SS-OCT system, where the spectral resolution of the system is changed in order to achieve different imaging depths. By simultaneous acquisition of a reference signal, which provides information about the swept source's wavenumber non-linearity, the linearly in time sampled data can be corrected for the non-linear wavenumber tuning of the source in time. As the sampling rate is in contrast to a k-clocked system not limited by the free spectral range (FSR) of the reference interferometer, one is able to achieve variable imaging depth, without modifying the k-clock.

### BRIEF DESCRIPTION OF THE FIGURES

FIG. 1 shows a generalized ophthalmic optical coherence tomography (OCT) system.

FIG. 2 shows reference signals and corresponding k-clock signals for two imaging modes. FIG. 2a shows a reference signal where the frequency swept laser is operated at 200 kHz. FIG. 2b shows the k-clock signal which was created on the fly by zero crossing detection electronics using the signal in FIG. 2a. FIG. 2c shows a reference signal where the swept laser is operated at 66 kHz, while maintaining the same optical path length difference as in FIG. 2a. Note that the x-axis is different in FIGS. 2a and 2c. FIG. 2d shows the k-clock signal which was created on the fly by zero crossing detection electronics using the signal in FIG. 2c. Note that the number of pulses per sweep is the same as in FIG. 2b but the frequency of the pulses is changed as indicated by the different time scales in the figures.

FIG. 3 shows reference signals and corresponding k-clock signals for two imaging modes with different k-clock signals. FIG. 3a shows a reference signal where the swept laser is operated at 200 kHz. FIG. 3b shows the k-clock signal which was created on the fly by zero crossing detection electronics using the signal in FIG. 3a. FIG. 3c shows a reference signal where the swept laser is operated at 66 kHz and the optical path length difference was increased as compared to FIG. 3a. Note that the x-axis is different in FIGS. 3a and 3c. FIG. 3d shows the k-clock signal which was created on the fly by zero crossing detection electronics using the signal in FIG. 3c. Note that the number of pulses per sweep is increased as compared to FIG. 3b.

FIG. 4 shows a SS-OCT system with a reference interferometer that can be used to collect information on the wavenumber non-linearity of the source to use in data resampling.

FIG. 5 shows reference signals and corresponding resampling vectors for two imaging modes. FIG. 5a shows a reference signal acquired while operating the swept laser at 200 kHz. The data acquisition rate was set to 500 MS/s. FIG. 5b shows the corresponding resampling vector calculated from the local phase of the signal shown in FIG. 5a. FIG. 5c shows a reference signal acquired while operating the swept laser at 66 kHz, while maintaining the same data acquisition rate and the optical path length difference as in 5a. FIG. 5d shows the corresponding resampling vector calculated from the local phase of the signal shown in FIG. 5c. Note that the resampling vector contains the same number of entries as the number of samples of the reference vector.

### DETAILED DESCRIPTION

A diagram of a generalized FD-OCT system for use in ophthalmology is shown in FIG. 1. Light from source 101 is routed, typically by optical fiber 105, to illuminate the sample 110, a typical sample being tissues in the human eye. The source 101 can be either a broadband light source with short temporal coherence length in the case of SD-OCT or a wavelength tunable laser source in the case of SS-OCT. The light is scanned, typically with a scanner 107 between the output of the fiber and the sample, so that the beam of light (dashed line 108) is scanned laterally (in x and y) over the area or volume to be imaged. Light scattered from the sample is collected, typically into the same fiber 105 used to route the light for sample illumination. Reference light derived from the same source 101 travels a separate path, in this case involving fiber 103 and retro-reflector 104 with an adjustable optical delay. Those skilled in the art recognize that a transmissive reference path can also be used and that the adjustable delay could be placed in the sample or reference arm of the interferometer. Collected sample light is combined with reference light, typically in a fiber coupler 102, to form light interference in a detector 120. An external clock can be part of the detector to control the sampling rate. Although a single fiber port is shown going to the detector, those skilled in the art recognize that various designs of interferometers can be used for balanced or unbalanced detection of the interference signal. The output from the detector is supplied to a processor 121. The results can be stored in the processor 121 or displayed on display 122.

The interference causes the intensity of the interfered light to vary across the spectrum. The Fourier transform of the interference light reveals the profile of scattering intensities at different path lengths, and therefore scattering as a function of depth (z-direction) in the sample (see for example Leitgeb et al. “Ultrahigh resolution Fourier domain optical coherence tomography,” Optics Express 12(10):2156 2004). The profile of scattering as a function of depth is called an axial scan (A-scan). A set of A-scans measured at neighboring locations in the sample produces a cross-sectional image (tomogram or B-scan) of the sample. A collection of B-scans makes up a data volume or cube scan. The range of wavelengths at which the interference is recorded (spectral range or bandwidth) determines the resolution with which one can determine the depth of the scattering centers, and thus the axial resolution of the tomogram. Recording a limited range of optical frequencies results in a coarser axial resolution.

The maximum imaging depth Δzₘₐₓ that one is able to achieve is related to the spectral resolution of the acquired data, δλ, according to: δλ=λ₀²/(2Δzₘₐₓ·n) with λ₀ being the central wavelength in the sweep and n being the average index of refraction of the medium to be imaged. In SS-OCT the spectral resolution is influenced by two factors, the instantaneous line width of the swept source and the number of sampling points per wavelength. The instantaneous line width of a swept source is typically a parameter, which may also be influenced by other parameters such as the sweep rate. The number of sampling points per wavelength is typically adjustable by either varying the sweep rate of the laser, while maintaining the same sampling frequency or alternatively varying the sampling frequency, while maintaining the same sweep rate of the laser.

In k-clocked SS-OCT systems the number of samples per sweep is commonly defined by the frequency of the k-clock and independent of the sweep rate of the laser. A common method to generate a k-clock is to output trigger pulses at zero crossing positions of a reference fringe signal. Those zero-crossing time points correspond to points linear in wavenumber. When one changes the sweep rate of the laser, the number of k-clock pulses per sweep remains the same as illustrated in FIG. 2. FIG. 2 shows reference signals and corresponding k-clock signals for two imaging modes. FIG. 2a shows a reference signal where the frequency swept laser is operated at 200 kHz. FIG. 2b shows the k-clock signal which was created on the fly by zero crossing detection electronics using the signal in FIG. 2a. FIG. 2c shows a reference signal where the swept laser is operated at 66 kHz, while maintaining the same optical path length difference as in FIG. 2a. Note that FIGS. 2a and 2c are shown on different x-axes (time scales). FIG. 2d shows the k-clock signal which was created on the fly by zero crossing detection electronics using the signal in FIG. 2c. Note that the number of pulses per sweep is the same as in FIG. 2b but the frequency of the pulses is changed as indicated by the different time scales in the figures. Consequently also the spectral resolution, defined by the number of sample points per sweep remains the same. Since the spectral resolution limits the imaging depth in SS-OCT, also the imaging depth remains the same. Therefore, if one would like to adjust the imaging depth of a k-clocked SS-OCT system one is forced to adjust the frequency of the k-clock. This involves mechanical or electrical changes as described in U.S. patent application Ser. No. 13/354,066 filed on Jan. 19, 2012.

FIG. 3 shows reference signals and corresponding k-clock signals for two imaging modes in which two different k-clocks are generated by changing the path length of the reference interferometer. FIG. 3a shows a reference signal where the swept laser is operated at 200 kHz. FIG. 3b shows the k-clock signal which was created on the fly by zero crossing detection electronics using the signal in FIG. 3a. FIG. 3c shows a reference signal where the swept laser is operated at 66 kHz and the optical path length difference was increased in the interferometer as compared to FIG. 3a. Note that FIGS. 3a and 3c are shown on different time scales or x-axes. FIG. 3d shows the k-clock signal which was created on the fly by zero crossing detection electronics using the signal in FIG. 3c. Note that the number of pulses per sweep is increased as compared to FIG. 3b. Collecting interferometric data with these two k-clocks would result in data at two different spectral resolutions and therefore could be used to generate images covering different imaging depths or ranges.

If one however uses an independent sampling clock for sampling the data and then resamples it using information of the swept source's wavenumber non-linearity as is proposed in the present invention, the sampling frequency is independent from a k-clock or other reference signal. One then has the freedom to resample to an arbitrary dense linear sampling in wavenumber. In this case the spectral resolution correlates to the number of output pixels after the resampling. The maximum achievable spectral resolution and therefore the maximum achievable imaging depth is limited by the spectral resolution of initial acquisition. One common approach for gaining information about the swept source's wavenumber non-linearity is to record, in parallel to the acquisition of the data from the imaging interferometer, the fringe signal of a reference interferometer (reference signal). Resampling describes the process of converting a digital signal from one sampling rate to another without changing acquisition parameters. In this particular application resampling describes the process of interpolating a digital signal, which was acquired at equally spaced points in time, at new sampling points which are equally spaced in wavenumber.

A swept-source OCT system with a reference interferometer is illustrated in FIG. 4. Compared to FIG. 1, the main difference is the introduction of a second fiber splitter 123 in the light source path that directs a portion of the light to a secondary interferometer with its own detector 124. The secondary interferometer illustrated in FIG. 4 is a fiber Mach Zehnder interferometer with a fixed path length mismatch. The output of the detector is directed to the processor 121. By collecting and extracting the unwrapped local phase of this signal using a Hilbert transformation along k one has direct access to the swept source's wavenumber non-linearity. Each of the entries of the resulting vector corresponds to a sample position linear in wavenumber.

FIG. 5 shows reference signals and corresponding resampling vectors for two imaging modes. FIG. 5a shows a reference signal acquired while operating the swept laser at 200 kHz. The data acquisition rate was set to 500 MS/s. FIG. 5b shows the corresponding resampling vector calculated from the local phase of the secondary interferometer signal shown in FIG. 5a. FIG. 5c shows a reference signal acquired while operating the swept laser at 66 kHz, while maintaining the same data acquisition rate and optical path length difference as in 5a. FIG. 5d shows the corresponding resampling vector calculated from the local phase of the signal shown in FIG. 5c. Note that the resampling vector contains the same number of positions as the number of samples of the reference vector for each case (2500 and 7500 respectively). Therefore these vectors may be conveniently used for resampling the acquired data to be sampled linear in wavenumber (see for example Yasuno et al. “Three-dimensional and high-speed swept-source optical coherence tomography for in vivo investigation of human anterior eye segments,” Opt. Express 13, 10652-10664, 2005). This information may be obtained from reference signals of arbitrary frequency. Changing the sweep rate of the laser, while maintaining the same sampling frequency does therefore not require a change of either the optical path length mismatch of the reference interferometer or an electronic multiplication or division of the reference signal's frequency.

The sampling of the reference signal is determined by the sampling frequency of the data acquisition device and is independent from the FSR of the reference interferometer. To that end the FSR need not be adjusted when collecting data at two different resolutions (e.g. change the path length of the reference interferometer) but the invention described herein is applicable when the FSR of the reference interferometer is changed or unchanged. In order to increase or reduce the number of samples of the reference signal, it may also be up- or down-sampled using interpolation methods prior to resampling. Using this approach one is able to vary the depth range of the system by adjusting the sweep rate of the laser, without requiring any changes of the reference interferometer or the system's electronics.

One is furthermore also able to adjust the imaging depth range by adjusting the sampling frequency of the data acquisition card. This can usually be done by adjusting the internal sampling clock frequency of the data acquisition card, independent from the reference's signal frequency. It is therefore possible to generate images of two different imaging depths by collecting data at two different spectral resolutions and resampling the data using information on the wavenumber nonlinearity of the source. Another method for gaining information about the swept source's wavenumber non-linearity is to capture the drive function of the swept source's tunable filter, which can then be used to determine the wavenumber non-linearity. Other techniques can be envisioned by those skilled in the art.

The invention may be used in all applications of swept source OCT that require variable imaging depth. In particular in ophthalmology, imaging of the anterior segment or posterior segment of the retina may require individually optimized imaging depth. Due to the curvature of the eye also different posterior segment imaging modes may require different imaging depths. A very wide field of view may e.g. require a larger imaging depth range than a relatively narrow field of view.

The following references are hereby incorporated by reference:

### PATENT REFERENCES

U.S. Pat. No. 7,375,818 Kawahara “Optical tomography system”
U.S. Pat. No. 7,602,500 Izatt et al. “Optical coherence imaging systems having a reduced effective linewidth and methods of using the same.”
U.S. Pat. No. 7,692,797 Kawahara “Optical tomography system”
US Publication No. 2007/0024856 Izatt et al “Optical coherence imaging systems having a reduced effective linewidth and methods of using the same”
US Publication No. 2010/0110376 Everett et al “Variable resolution optical coherence tomography scanner and method for using same”
US Publication No. 2010/0150422 Vakoc et al. “Systems and methods for extending imaging depth range of optical coherence tomography through optical sub-sampling”
Publication No. WO 2010/006785 Hacker et al “Optical coherence tomography methods and systems”
Publication No. WO 2011/037980 Buckland et al “Systems for extended depth frequency domain optical coherence tomography (FDOCT) and related methods”
U.S. patent application Ser. No. 13/354,066 filed on Jan. 19, 2012

### NON-PATENT REFERENCES

Golubovic et al. “Optical frequency-domain reflectometry using rapid wavelength tuning of a Cr⁴⁺:fosterite laser” Optics Letters 22(22), 1704-1706 (1997).
Gora et al “Ultra high-speed swept-source OCT imaging of the anterior segment of human eye at 200 kHz with adjustable imaging range,” Optics Express 17(17):14880 (2009).
Huber et al. “Amplified, frequency swept laser for frequency domain reflectometry and OCT imaging: design and scaling principles,” Optics Express 13(9), 3513-3528 (2005).
Lee et al. “Wide tuning range wavelength-swept laser with a single SOA at 1020 nm for ultrahigh resolution Fourier-domain optical coherence tomography” Optics Express 19(22) 21227 (2011).
Lee et al. “Optimization for axial resolution, depth range, and sensitivity of spectral domain optical coherence tomography at 1.3 microns” Journal of Korean Physical Society 55(6): 2354-2360 (2009).
Leitgeb et al. “Ultrahigh resolution Fourier domain optical coherence tomography,” Optics Express 12(10):2156 (2004).
Xi et al “Generic real-time uniform K-space sampling method for high-speed swept-source optical coherence tomography,” Optics Express 18(9):9511 (2010).
Yasuno et al. “Three-dimensional and high-speed swept-source optical coherence tomography for in vivo investigation of human anterior eye segments,” Opt. Express 13, 10652-10664 (2005).
Yun et al. “High-speed optical frequency-domain imaging” Optics Express 11(22), 2953-2963 (2003).

What is claimed is:

1. A method for generating optical coherence tomography (OCT) images covering two different depth ranges in a sample of interest, said method comprising:
collecting a series of OCT data over a series of transverse location in the eye, wherein the data is collected using a source that is swept in frequency and the data is acquired at two different spectral resolutions;
collecting information on the nonlinearity of the source's wavenumber values during the sweep for each spectral resolution;
resampling the OCT data for each spectral resolution based on the collected information so that the data is linear in wavenumber;
processing the resampled data at one spectral resolution to generate an image covering one depth range in the eye;
processing the resampled data at the second spectral resolution to generate an image covering a second depth range in the eye; and
storing or displaying the processed images.

2. A method as recited in claim 1, wherein the information on the wavenumber nonlinearity is recorded using a reference interferometer.

3. A method as recited in claim 2, wherein the free spectral range of the reference interferometer is modified when collecting the data at two different spectral resolutions.

4. A method as recited in claim 2, wherein the free spectral range of the reference interferometer is not modified when collecting the data at two different spectral resolutions.

5. A method as recited in claim 1, wherein the signal containing information of the source's wavenumber values during the sweep is not modified when collecting the data at two different spectral resolutions.

6. A method as recited in claim 1, wherein the signal containing information of the source's wavenumber values during the sweep is modified when collecting the data at two different spectral resolutions.

7. A method as recited in claim 1, wherein the sweep speed of the source is adjusted to collect the data at two different spectral resolutions.

8. A method as recited in claim 1, wherein the data is acquired using a k-clock.

9. A method as recited in claim 1, wherein the information on the wavenumber nonlinearity is collected from the drive function of the source.

10. A method as recited in claim 1, wherein the information of the source's wavenumber values during the sweep is processed prior to using it for resampling.

11. A method as recited in claim 10, wherein the information of the source's wavenumber values during the sweep is interpolated in order to up-sample the information.

12. A method as recited in claim 10, wherein the information of the source's wavenumber values during the sweep is interpolated in order to down-sample the information.

13. A method as recited in claim 1, wherein the information of the source's wavenumber values during the sweep is used to resample the OCT data to a different number of samples compared to its original number of samples.

14. A method as recited in claim 10, wherein the processed information of the source's wavenumber values during the sweep is used to resample the OCT data to a different number of samples compared to its original number of samples.

15. A method for generating images of the eye of a patient using an optical coherence tomography (OCT) system, said system including a light source generating an output that is swept in frequency over time, said system further including a reference interferometer, said method comprising:
collecting a first set of OCT data over a series of transverse location in the eye, while the light source is swept in frequency at a first sweep rate;
collecting a second set of OCT data over a series of transverse location in the eye, while the light source is swept in frequency at a second sweep rate different from the first sweep rate;
resampling the first set of OCT data based on information generated by the reference interferometer while the first set of OCT data is being collected;
processing the resampled first set of OCT data to generate an image covering a first depth range in the eye;
resampling the second set of OCT data based on information generated by the reference interferometer while the second set of OCT data is being collected;
processing the resampled second set of OCT data to generate an image covering a second depth range in the eye; and
storing or displaying the processed images.

16. A method as recited in claim 15 wherein the rate the OCT data is sampled is the same during the collection of the first and second sets of OCT data.
